(19) United States
(12) Patent Application Publication
(10) Pub. No.: US 2010/0087481 A1
(43) Pub. Date: Apr. 8, 2010

(54) ORAL PHARMACEUTICAL FORMULATIONS FOR ANTIDIABETIC COMPOUNDS

(76) Inventor: Kathleen M. Lee, Palo Alto, CA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/572,803

(22) Filed: Oct. 2, 2009

Related U.S. Application Data
(60) Provisional application No. 61/102,658, filed on Oct. 3, 2008.

(51) Int. Cl. A61K 31/47; A61P 3/10

(52) U.S. Cl. 514/312

(57) ABSTRACT

Oral pharmaceutical preparations of salts and polymorphs of a compound useful in the treatment of inflammatory and metabolic conditions and diseases are provided herein. The oral pharmaceutical preparation is useful for the treatment or prevention of conditions and disorders associated with energy homeostasis such as type II diabetes, lipid metabolism, adipocyte differentiation and inflammation.

### 1. FIELD OF THE INVENTION

Oral pharmaceutical preparations are provided herein for the administration of certain modulators of the peroxisome proliferator-activated receptor γ (“PPARγ”) receptor. The oral pharmaceutical preparations comprise a therapeutically effective amount of a salt form of the PPARγ modulators in an oil-based medium, preferably the active ingredient and oil-based medium are encapsulated, e.g., in a capsule. Also provided are methods of making the oral pharmaceutical preparations and methods of their use for the treatment of, for example, type II diabetes (and complications thereof), hypercholesterolemia (and related disorders associated with abnormally high or low plasma lipoprotein or triglyceride levels) and inflammatory disorders.

### 2. BACKGROUND OF THE INVENTION

Peroxisome proliferator-activated receptor γ (“PPARγ”) is one member of the nuclear receptor superfamily of ligand-activated transcription factors and has been shown to be expressed in an adipose tissue-specific manner. Its expression is induced early during the course of differentiation of several preadipocyte cell lines. Additional research has now demonstrated that PPARγ plays a pivotal role in the adipogenic signaling cascade. PPARγ also regulates the ob/leptin gene which is involved in regulating energy homeostasis and adipocyte differentiation, which has been shown to be a critical step to be targeted for treating disorders such as obesity, diabetes and dyslipidemia.

In view of the clinical importance of PPARγ compounds that modulate PPARγ function can be used for the development of new therapeutic agents. Potent modulators of PPARγ have been described, for example, in International Patent Publication No. WO 01/00579, and U.S. Pat. No. 6,200,995 B1, U.S. Pat. No. 6,583,157 B2, U.S. Pat. No. 6,653,332, and U.S. Pat. No. 7,041,691 B1. One of these promising modulators, identified herein as compound 101, is in clinical development for therapeutic treatment of type II diabetes. A suitable formulation or dosage form for this molecule may be essential for its use in the prevention or treatment of disease. Indeed, formulations that improve stability or increase bioavailability would be particularly useful.

The free base and certain pharmaceutically acceptable salts of compound 101 are described in International Patent Publication No. WO 01/00579, and U.S. Patent No. 6,583,157 B2 and U.S. Pat. No. 7,041,691. U.S. Patent No. 7,223,761 B2 discloses that the besylate salt of compound 101, and polymorphs thereof, displays superior stability and hygroscopic properties when compared to other salts of compound 101. Despite the superior stability and hygroscopic properties of the besylate salt of compound 101, the besylate salt remains sparingly soluble in aqueous solvents and in most organic solvents, which limits its effective concentration in a pharmaceutical preparation, which in turn can lead to decreased bioavailability upon administration. A need therefore exists for improved formulations of oral pharmaceutical preparations of besylate and other salt and polymorphic forms of this class of PPAR modulators in the treatment of inflammatory and metabolic conditions and diseases which display improved solubility and shelf-life stability. These and other unmet needs are addressed by this disclosure.

### 3. SUMMARY OF THE INVENTION

Provided herein are oral pharmaceutical preparations of a PPARγ modulator which are useful in the treatment or prevention of conditions and disorders including but not limited to those associated with energy homeostasis, lipid metabolism, adipocyte differentiation, inflammation, and diabetic conditions, such as, for example, hyperglycemia and hyperinsulemia.

In certain embodiments, the oral pharmaceutical preparations provided herein display surprisingly good solubility and shelf-life stability, which would render them particularly suitable for the treatment or prevention of the conditions and disorders disclosed herein.

In certain embodiments, the surprisingly good solubility of the oral pharmaceutical preparations allows for a higher concentration of the active ingredient in a smaller volume.

In one aspect, provided herein is an oral pharmaceutical preparation comprising a dissolved form of a PPARγ modulator, in an oil-based medium, optionally in a capsule. In one aspect, the PPARγ modulator is added in a solid form during the manufacturing process and is dissolved in an oil-based medium.

In certain aspects, provided herein is an oral pharmaceutical preparation comprising: (a) a PPARγ modulator, or salt thereof, in an oil-based medium; and (b) a capsule encapsulating the composition.

In certain embodiments, the PPARγ modulator has the following structure (I):

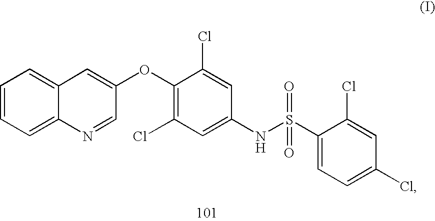

which is described in International Patent Publication No. WO 01/00579, and in U.S. Patent No. 6,583,157 B2 and U.S. Pat. No. 7,041,691 B1, the contents of which are hereby incorporated by reference in their entireties.

In preferred embodiments, provided herein are stable pharmaceutical preparations of the benzenesulfonic acid salts of compound 101, wherein said preparations are useful for oral administration.

In further aspects, provided herein are polymorphs of the benzenesulfonic acid salts of compound 101, identified as Form I and Form II, each described in detail below.

In certain embodiments, the oral pharmaceutical preparations comprising the benzenesulfonic acid salts and polymorphs of compound 101 display surprisingly good solubility in oil-based media.

In certain embodiments, the oral pharmaceutical preparations comprising the benzenesulfonic acid salts and polymorphs of compound 101 display surprising physical and chemical stability.

In certain embodiments, the oral pharmaceutical preparations comprising the benzenesulfonic acid salts and polymorphs of compound 101 display no visual precipitation, no leakage, no degradation, or no loss of potency when stored for over 12 months at 25° C./60% RH.

In certain embodiments, the oil-based medium of the pharmaceutical preparations disclosed herein comprises corn oil, super-refined soybean oil, Capmul® MCM, Capmul® PG8, Captex® 200, Captex® 300 EP, Captex® 355, Crodamol®, Inwitor 742, Labrafac® CC, Labrafil® M 1944 CS, Labrasol®, Peceol, Phosal® 53 MCT, Phospholipon® 90G, Maisine Miglyol® 132, Miglyol® 810N, Miglyol® 812N, or mixtures thereof. In preferred embodiments, the oil-based medium comprises Miglyol® 812N and Phospholipon® 90 G in a wt/wt ratio of about 60:40 Miglyol® 812N to Phospholipon® 90 G.

In certain embodiments, the oral pharmaceutical preparations disclosed herein are encapsulated in a capsule for oral delivery. In preferred embodiments, the capsule comprises hard gelatin. In other preferred embodiments, the capsule is sealed using gelatin banding.

In certain embodiments, the gelatin banding prevents leakage of the contents of the capsule.

In another aspect, provided herein are methods for the treatment or prevention of a condition or disorder mediated by the PPARγ receptor, comprising administering to a subject in need of such treatment or prevention an oral pharmaceutical preparation as described herein.

In certain embodiments, the PPARγ-mediated condition or disorder is a metabolic disorder or an inflammatory condition. In certain embodiments, the metabolic disorder is selected from the group consisting of diabetes, obesity, hypercholesterolemia, hyperlipidemia, dyslipidemia, hypertriglyceridemia, hyperglycemia, insulin resistance and hyperinsulinemia. In preferred embodiments, the metabolic disorder is type II diabetes. In certain embodiments, the inflammatory condition is selected from the group consisting of rheumatoid arthritis and atherosclerosis. In certain embodiments, the subject is human.

In another aspect, provided herein are methods of making an oral pharmaceutical preparation as described herein.

### 4. DETAILED DESCRIPTION OF THE INVENTION

### 4.1 Definitions

The terms “treat,” “treating” or “treatment,” as used herein, refer to a method of alleviating or abrogating a disease and/or its attendant symptoms. The teems “prevent,” “preventing” or “prevention,” as used herein, refer to a method of administration prior to onset of disease or manifestation of its symptoms. In certain embodiments, “prevent,” “preventing” or “prevention,” as used herein, refers to delaying the onset of disease or manifestation of its symptoms.

As used herein, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, “diabetes” refers to type I diabetes mellitus (juvenile diabetes) or type II diabetes mellitus (non-insulin-dependent diabetes mellitus or NIDDM), preferably, type II diabetes mellitus.

As used herein, the term “PPARγ-mediated condition or disorder” or “PPARγ-mediated condition or disease,” and the like, refers to a condition, disorder, or disease characterized by inappropriate, e.g., less than or greater than normal, PPARγ activity. Inappropriate PPARγ activity might arise as the result of PPARγ expression in cells which normally do not express PPARγ, increased PPARγ expression (leading to, e.g., certain energy homeostasis, lipid metabolism, adipocyte differentiation and inflammatory disorders and diseases), or, decreased PPARγ expression (leading to, e.g., certain energy homeostasis, lipid metabolism, adipocyte differentiation and inflammatory disorders and diseases). A PPARγ-mediated condition or disorder may be completely or partially mediated by inappropriate PPARγ activity. However, a PPARγ-mediated condition or disorder is one in which modulation of PPARγ results in some effect on the underlying condition or disease (e.g., a PPARγ modulator results in some improvement in patient well-being in at least some patients). Exemplary PPARγ-mediated conditions and disorders include, but are not limited to, metabolic disorders, e.g., diabetes, type II diabetes, obesity, hyperglycemia, insulin resistance, hyperinsulinemia, hypercholesterolemia, hypertension, hyperlipoproteinemia, hyperlipidemia, hypertriglyceridemia and dyslipidemia, and inflammatory conditions, e.g., rheumatoid arthritis and atherosclerosis.

The term “modulate,” in its various forms, refers to the ability of a compound to increase or decrease the function or activity associated with a particular peroxisome proliferator-activated receptor, preferably the PPARγ receptor. Modulation, as described herein, includes the inhibition or activation of PPARγ, either directly or indirectly. Inhibitors are compounds that, e.g., bind to, partially or totally block stimulation, decrease, prevent, delay activation, inactivate, desensitize, or down regulate signal transduction, e.g., antagonists. Activators are compounds that, e.g., bind to, stimulate, increase, open, activate, facilitate, enhance activation, sensitize or up regulate signal transduction, e.g., agonists. Further, modulation of PPARγ receptor activity is intended to encompass antagonism, agonism, partial antagonism and/or partial agonism of the activity associated with the PPARγ receptor. Modulation may also include partial agonism and partial antagonism of the activity associated with the PPARγ receptor by some modulator; i.e., a modulator with partial agonist and antagonist activity.

By “pharmaceutically acceptable” it is meant the active ingredient, salt, polymorph, diluent, excipient or carrier must be compatible with the other ingredients of the formulation and not unduly deleterious to the recipient thereof.

The term “therapeutically effective amount” refers to the amount of the subject compound, including a salt or polymorph of the compound, that will elicit the biological or medical response of a tissue, system, animal or human that is being sought by the researcher, veterinarian, medical doctor or other clinician or that is sufficient to prevent development of or alleviate to some extent one or more of the symptoms of the disease being treated.

The term “subject” is defined herein to include animals such as mammals, including, but not limited to, primates (e.g., humans), cows, sheep, goats, horses, dogs, cats, rabbits, rats, mice, and the like. In preferred embodiments, the subject is a human.

The term “microemulsion” means a clear, stable, isotropic liquid mixture of oil, water, and surfactant, frequently in combination with a cosurfactant. The aqueous phase may contain salt(s) and/or other ingredients, and the “oil” may actually be a complex mixture of different hydrophobic components, e.g., hydrocarbons and olefins.

The term “salt” or “salts” is meant to include acid-base ionic complexes of active compounds which are prepared by reacting an acid of the active compound with a relatively nontoxic base, or a base of the compound with a relatively nontoxic acid. Acid addition salts can be obtained by contacting the neutral faun of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like. (See, for example, Berge et al., 1977, J. Pharm. Sci. 66:1-19.)

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound.

The term “solid forms” and related terms used herein, unless otherwise specified, refers to crystalline forms and amorphous forms comprising compound 101 and its various salt forms.

The term, “crystalline,” and related terms used herein, when used to describe a substance, component or product, means that the substance, component or product is crystalline as determined by X-ray diffraction. See, e.g., Remington's Pharmaceutical Sciences, 18^{th} ed., Mack Publishing, Easton Pa., 173 (1990); The United States Pharmacopeia, 23^{rd} ed., 1843-1844 (1995).

The term “crystalline forms” and related terms herein refers to the various crystalline modifications of a given substance, including, but not limited to, polymorphs, solvates, hydrates, co-crystals and other molecular complexes, as well as salts, solvates of salts, hydrates of salts, other molecular complexes of salts, and polymorphs thereof.

The terms, “polymorphs” and “polymorphic forms” and related terms herein refer to crystalline forms of the same molecule. Different polymorphs may have different physical properties such as, for example, melting temperatures, heats of fusion, solubilities, dissolution rates, and/or vibrational spectra as a result of the arrangement or conformation of the molecules in the crystal lattice.

As used herein, a salt or polymorph that is “pure,” i.e., substantially free of other polymorphs, contains less than about 10% of one or more other polymorphs, preferably less than about 5% of one or more other polymorphs, more preferably less than about 3% of one or more other polymorphs, most preferably less than about 1% of one or more other polymorphs.

The term, “solvate,” as used herein, refers to a solid form of a substance which contains solvent. The term “hydrate” refers to a solvate wherein the solvent is water.

The term, “amorphous form,” as used herein, refers to a noncrystalline form of a substance.

### 4.2 Embodiments of the Invention

Provided herein are storage stable pharmaceutical compositions suitable for oral delivery of PPARγ compounds which compositions provide improved solubility over known preparations of the compound. These stable oral dosage forms provide sufficient bioavailability while preserving stable storage life such that they are uniquely suited for use for delivery of the PPARγ compounds.

In certain embodiments, provided herein are oral pharmaceutical preparations of PPARγ compounds. In certain embodiments, the oral pharmaceutical preparations comprise compound 101, including the salt forms and polymorphs of compound 101, in an oil-based medium, encapsulated in a capsule for oral delivery.

In certain embodiments, provided herein are methods of making the oral pharmaceutical preparations and methods of use in the modulation of receptor activity, particularly PPARγ activity.

Preferred salts and polymorphs of compound 101 for use in the oral pharmaceutical preparations are the Form I and Form II polymorphs of the besylate salt of compound 101.

Preferred oil-based media of the oral pharmaceutical preparations are those which increase solubility of the salt forms and polymorphs, which form stable microemulsions, which prevent the formation of precipitates, and which contribute to the chemical and physical stability of the oral pharmaceutical preparations.

Preferred capsules of the oral pharmaceutical preparations are those which display improved stability and decreased oxygen permeability, which prevent leakage of the contents of the capsule, and/or which protect the contents of the capsule from heat and humidity.

The oral pharmaceutical preparations can be used in the treatment or prevention of conditions and disorders associated with diabetic conditions, energy homeostasis, lipid metabolism, adipocyte differentiation and inflammation. (See, Ricote et al., 1988, Nature 391:79-82, and Jiang et al., 1998, Nature 391:82-86.) For example, the oral pharmaceutical preparations can be useful in the treatment of metabolic disorders, such as type II diabetes. Additionally, the oral pharmaceutical preparations can be useful for the prevention and treatment of complications of metabolic disorders, such as type II diabetes, e.g., neuropathy, retinopathy, glomerulosclerosis, and cardiovascular disorders.

4.2.1 Oral Pharmaceutical Preparations

In one aspect, provided herein are oral pharmaceutical preparations of a PPARγ modulator useful in the treatment of inflammatory and metabolic conditions and diseases. In some embodiments, the oral pharmaceutical preparations comprise a salt or polymorph of compound 101, as discussed below. In some embodiments, the salts and/or polymorphs of the oral pharmaceutical preparations can be in an oil-based medium, as discussed below. In some embodiments, the salts and/or polymorphs of the oral pharmaceutical preparations can be in a capsule, as discussed below. In some embodiments, the oral pharmaceutical preparations can further comprise other additives, as discussed below.

4.2.1.1 Salts of Compound 101

Pharmaceutically acceptable salts of compound 101, a potent modulator of the PPARγ receptor, having particular utility for the treatment or prevention of conditions and disorders associated with energy homeostasis, lipid metabolism, adipocyte differentiation, inflammation, and diabetes or diabetic conditions that can be used in the pharmaceutical compositions or methods provided herein include but are not limited to HCl, HBr, tosylate, and besylate salts of compound 101.

In preferred embodiments, besylate salts of compound 101 are used within the methods and compositions. A preferred besylate salt of compound 101 is provided by formula (I):

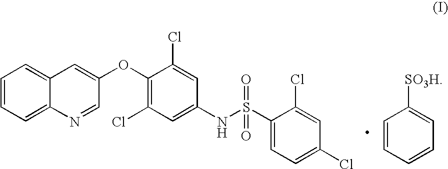

Each salt provided herein can be made from a preparation of compound 101, which can be synthesized or obtained according to any method apparent to those of skill in the art. In certain embodiments, compound 101 is prepared according to the methods described in detail in the examples below, and in U.S. Pat. No. 6,583,157 B2 and U.S. Pat. No. 7,223,761 B2, the contents of which are hereby incorporated by reference in their entireties.

4.2.1.2 Polymorphs of Compound 101

Also useful within the compositions and methods are polymorphs of compound 101. In certain embodiments, the polymorphs are polymorphs of the besylate salt of compound 101 described above. In certain embodiments, the polymorphs can be pure polymorphs of the besylate salt of compound 101. For example, a polymorph can be a pure Form I polymorph or a pure Form II polymorph of the besylate salt of compound 101.

The polymorphs of the HCl, HBr, tosylate, and besylate salts of compound 101 have been extensively characterized and described in U.S. Pat. No. 7,223,761 B2. Certain details regarding the Form I and Form II polymorphs of the besylate salt of compound 101 are reproduced below.

Each polymorph can be made from a preparation of compound 101. Solid compound 101 can be dissolved and then crystallized from the solvent mixtures described below to yield the polymorphic forms described herein. In particular embodiments, provided herein is a besylate salt of compound 101 which can be dissolved and then crystallized from the solvent mixtures described below to yield the polymorphic forms described herein.

In one embodiment, provided herein is a Form I of a besylate salt of compound 101 (2,4-Dichloro-N-[3,5-dichloro-4-(quinolin-3-yloxy)-phenyl]-benzenesulfonamide benzenesulfonate salt). In one embodiment, the Form I polymorph of the besylate salt of compound 101 has a melting point of about 180° C. or greater. In a particular embodiment, the Form I polymorph has a melting point between about 180 and 200° C. When an exemplary Form I polymorph was examined by differential scanning calorimetry according to the methods described in the examples below, it had an endotherm at between about 186.3° C. and about 189.5° C., and an enthalpy of fusion of between about 81.5 J/g and about 89.9 J/g. In further embodiments, the Form I polymorph of the besylate salt of compound 101 has major X-ray powder diffraction pattern peaks at 7.0, 19.5, 22.0, 24.0, 24.5, and 28° 2θ using Cu Ka radiation. In certain embodiments, the Form I polymorph described herein has major X-ray powder diffraction pattern peaks at one, two, three, four, five or six of the X-ray powder diffraction pattern peaks at 7.0, 19.5, 22.0, 24.0, 24.5, and 28° 2θ using Cu Ka radiation. In further embodiments, the Form I polymorph described herein has both a melting point between about 186 and 200° C. and major X-ray powder diffraction pattern peaks at one, two, three, four, five or six of the X-ray powder diffraction pattern peaks at 7.0, 19.5, 22.0, 24.0, 24.5, and 28° 2θ using Cu Ka radiation. In still further embodiments, the Form I polymorph described herein has major infrared absorbance peaks at one, two, three, four, or five of the infrared absorbance peaks at 1567, 1461, 913, 895, and 881 cm⁻¹.

Form I of the besylate salt of compound 101 can be made by any method of making Form I apparent to those of skill in the art based upon the teachings herein. In certain embodiments, Form I can be crystallized from ethanol solutions of compound 101 and a hydrate of benzenesulfonic acid. Preferably, an ethanol solution of benzenesulfonic acid hydrate (Aldrich) can be added to solid compound 101 under heat to complete solution; cooling the solution yields Form I. Form I can also be crystallized from solutions of ethyl acetate and ethanol as described in the examples below.

In another embodiment, provided herein is a Form II of the besylate salt of compound 101 (2,4-Dichloro-N-[3,5-dichloro-4-(quinolin-3-yloxy)-phenyl]-benzenesulfonamide benzenesulfonate salt). In one embodiment, the Form II polymorph of the besylate salt of compound 101 has a melting point of about 230° C. or greater. In a particular embodiment, the Form II polymorph has a melting point between about 230 and 240° C. An exemplary Form II of the besylate salt of compound 101 displayed advantageous stability and had a melting temperature of about 233° C. When an exemplary Form II polymorph was examined by differential scanning calorimetry according to the methods in the examples below, it had an endotherm at about 233.7° C. and an enthalpy of fusion of about 98.9 J/g. In further embodiments, the Form II polymorph of the besylate salt of compound 101 has major X-ray powder diffraction pattern peaks at 15, 19, 20.5, 23.5, 24.5, 25, 26.5, 29.5, and 30.5° 2θ using Cu Ka radiation. In certain embodiments, the Form II polymorph provided herein has major X-ray powder diffraction pattern peaks at one, two, three, four, five, six, seven or eight of the X-ray powder diffraction pattern peaks at 15, 19, 20.5, 23.5, 24.5, 25, 26.5, 29.5, and 30.5° 2θ using Cu Ka radiation. In certain embodiments, the Form II polymorph provided herein has both a melting point between about 230 and 240° C. and major X-ray powder diffraction pattern peaks at one, two, three, four, five, six, seven or eight of the X-ray powder diffraction pattern peaks at 15, 19, 20.5, 23.5, 24.5, 25, 26.5, 29.5, and 30.5° 2θ using Cu Ka radiation. In further embodiments, the Form II polymorph provided herein has major infrared absorbance peaks at one, two, three, four, or five of the infrared absorbance peaks at 1573, 1469, 1459, 912, and 859 cm^{-l}.

Form II of the besylate salt of compound 101 can be made by any method apparent to those of skill in the art to make Form II based upon the teachings herein. In certain embodiments, Form II can be crystallized from solutions of ethyl acetate and ethanol as described in the examples below. Preferably, Form II of the besylate salt of compound 101 can be prepared by adding an ethanol solution of benzenesulfonic acid to solid compound 101 under heat. The reaction suspension can be stirred under heat, then cooled under further stirring, which yields Form II of the besylate salt of compound 101.

In certain embodiments, provided herein are Form I or II of a besylate salt of compound 101 obtained by crystallization of either of Forms I or II of the besylate salt of compound 101 and conversion of the crystallized form to the other form (e.g., crystallization of Form I and conversion of Form I to Form II) in solution or in the solid state.

As described in detail in U.S. Pat. No. 7,223,761 B2, the besylate salt of compound 101 exhibits superior properties to other acid addition salts of compound 101. The Form I and Form II polymorphs of the besylate salt of compound 101, and polymorphs thereof, display advantageous stability and hygroscopicity for use in a formulation for administration to animals or humans. Form II of the besylate salt of compound 101 is preferred over Form I of the besylate salt of compound 101 because of its greater stability.

4.2.1.3 Oil-Based Media

In certain embodiments, the oral pharmaceutical preparations provided herein comprise an oil-based medium. In certain embodiments, the composition comprising the oil-based medium encompasses compositions that have the effect of increasing the solubility of the salt or polymorph of compound 101 in the oil-based medium.

In certain embodiments, certain combinations of the oil-based medium provide surprisingly advantageous properties. For example, in certain embodiments, certain combinations of the oil-based medium provide surprising solubility of compound 101 or its salts or polymorphs. In certain embodiments, certain combinations of the oil-based medium provide surprising physical and chemical stability which may contribute to improved shelf-life.

In certain embodiments, the oil-based medium comprises a vegetable oil. The oil-based medium may comprise, for example, corn oil or super-refined soybean oil.

In certain embodiments, the oil-based medium comprises a lipid excipient, a solubilizing agent, a surfactant, a co-surfactant, an emulsifier, or a dispersing or wetting agent. The oil-based medium may comprise, for example, one or more members selected from the following: Capmul® MCM, Capmul® PG8, Captex® 200, Captex® 300 EP, Captex® 355, Crodamol®, Inwitor 742, Labrafac® CC, Labrafil® M 1944 CS, Labrasol®, Peceol, Phosal® 53 MCT, Phospholipon® 90G, Maisine 35-I, Miglyol® 132, Miglyol® 810N, and Miglyol® 812N, or any other oil-based medium known to one of skill in the art.

In certain embodiments, the oil-based medium comprises one or more members selected from the group above.

In certain embodiments, the oil-based medium comprises two or more members selected from the group above.

In certain embodiments, the oil-based medium comprises a phosphatidylcholine, for example, Phospholipon® 90 G.

In certain embodiments, the oil-based medium comprises a caprylic/capric acid, for example, Capmul® MCM, Capmul® PG8, Captex® 200, Captex® 300 EP, Captex® 355, Crodamol®, Inwitor 742, Labrafac® CC, Labrasol®, Miglyol® 132, Miglyol® 810N, or Miglyol® 812N.

In certain embodiments, the oil-based medium comprises a caprylic/capric acid and a phosphatidylcholine.

In particular embodiments, the oil-based medium comprises Miglyol® 812N and Phospholipon® 90 G. In preferred embodiments, the oil-based medium comprises Miglyol® 812N and Phospholipon® 90 G in a wt/wt ratio of about 60:40 Miglyol® 812N to Phospholipon® 90 G.

The pharmaceutical compositions may also be in the form of a microemulsion. In preferred embodiments, the microemulsion remains a clear, stable, isotropic liquid at room temperature, for an extended storage time of two or more years.

4.2.1.4 Capsules

4.2.1.4.1 Capsule Materials

In certain embodiments, the oral pharmaceutical preparations provided herein comprise a device which encapsulates the salts and/or polymorphs of compound 101 in the oil-based medium. In certain embodiments, the device may be a capsule, an enteric-coated tablet, or an enteric-coated caplet.

In certain embodiments, the device is a capsule.

In certain embodiments, the capsule comprises gelatin, plasticized gelatin, hydroxypropylmethylcellulose, starch or agar, or any other material known to one of skill in the art.

In certain embodiments, the capsule comprises gelatin.

In certain embodiments, the capsule comprises soft gelatin.

In preferred embodiments, the capsule comprises hard gelatin.

Hard capsules can be produced from the capsule material using conventional techniques known to those of ordinary skill in the art and described in, for example, U.S. Patent No. 4,917,885.

Plasticizers may be added to the capsule material to increase the flexibility and strength and may be selected from glycerin, propylene glycol, polyethylene glycol, triethyl citrate, acetyl triethyl citrate, diethyl phthalate, dibutyl phthalate, dibutyl sebacate, or mixtures thereof, or any other material or mixture known to one of skill in the art. The plasticizer may be present in an amount ranging from 0.1% to 30% by weight of the capsule.

The capsules described herein can be sized to hold the desired amount of a formulation, typically up to about 450-500 mg of a formulation. However, the preferred amount of a formulation in the capsule is about 350 mg. Preferably, the size of any particular capsule described herein will correspond to a conventional capsule size, e.g. Size Nos. 00, 0, 1, 2, 3, 4, 5, and the like. (See, for example, Remington's The Science and Practice of Pharmacy, 21st Ed (2005).)

In preferred embodiments, the capsule size is Size No. 1.

The filling of the contents in the capsules can be performed using any capsule-filling technique known to those skilled in the art.

4.2.1.4.2 Sealing and Banding

Once the capsule body is filled with a pharmaceutical formulation, and the capsule cap is secured onto the capsule body to form a liquid-filled capsule, the capsule may optionally be sealed. Any capsule-sealing technique known to those skilled in the art may be used. Advantageously, sealed capsules show evidence of tampering; they may also thwart efforts of individuals attempting to tamper with the capsule contents. In addition, sealed capsules safeguard against contaminants entering the capsule interior, and/or also prevent leakage of the formulation from the capsule interior.

In certain embodiments, the capsule body and cap are sealed at their seam of overlap using a sealing fluid. The sealing fluid may be applied by, for example, spraying the capsule with the sealing fluid which is directed to the overlap region.

The sealing fluid may comprise one or more of, for example, an organic solvent or an aqueous solution of an organic solvent, which depress the melting point of gelatin. In certain embodiments, the sealing fluid is ethanol.

In preferred embodiments, the joined capsule halves are banded at their seam of overlap with a gelatin band. The gelatin band may be placed around the capsule in a solution form and then cooled, thereby sealing the capsule.

In certain embodiments, the gelatin band is applied as a gelatin banding solution.

In certain embodiments, the gelatin banding solution contains 21.69% gelatin, 0.92% polysorbate 80 NF/PhEur/JP, 77.39% purified water.

In certain embodiments, the gelatin band prevents leakage of the contents of the capsule.

4.2.1.4.3 Enteric Coating

The capsules of the oral pharmaceutical preparations provided herein may also be coated with an enteric coating, alone or in addition to another coating. Enteric coating of oral pharmaceutical preparations that contain drugs is well known in the pharmaceutical sciences literature. (See, for example, Remington's Pharmaceutical Sciences, 19th Ed. (1990), the content of which is incorporated in its entirety.)

The enteric materials for use in the enteric coating preferably prevent release of the enteric-coated drug in gastric fluid of the stomach and prevent exposure of the drug to the acidity of the gastric contents while the enteric coated drug composition is in the stomach. After passing from the stomach into the intestine, the enteric coating preferably dissolves and releases the drug into intestinal fluids.

Materials suitable for use in the enteric coating include hydroxypropyl methylcellulose, hydroxyethyl cellulose, hydroxypropyl cellulose, methylcellulose, ethylcellulose, acrylic acid methacrylic acid ester copolymer, or a mixture thereof.

Additional materials suitable for use in the enteric coating include phthalates including hydroxypropyl methylcellulose phthalate, hydroxyethyl cellulose phthalate, hydroxypropyl cellulose phthalate, methylcellulose phthalate, ethylcellulose phthalate, and cellulose acetate phthalate.

In other embodiments of the oral pharmaceutical preparations provided herein, the capsule may additionally be coated with a controlled release coating, which is compatible with the other components of the enteric coating. The controlled release coating may comprise a hydrophobic controlled release material selected from an alkylcellulose, an acrylic polymer, or mixtures thereof.

In certain embodiments, the controlled release coatings include a plasticizer such as those described herein.

4.2.1.5 Other Additives

The oral pharmaceutical preparations for the administration of the salts or polymorphs provided herein may comprise other additives. For example, the oral pharmaceutical preparations provided herein may further comprise a solvent (e.g., ethanol), a stabilizer, a binder, a filler, a surfactant, a preservative, an antioxidant, a wetting or emulsifying agent, a suspending or dispersing agent, an inert gas, a sweetening agent, a flavoring agent, a coloring agent, or a mixture thereof, or any other additive known to one of skill in the art to provide pharmaceutically elegant and palatable preparations.

The oral pharmaceutical preparations provided herein may further comprise other suitable agents for combination therapy as noted herein, which are usually applied in the treatment or prevention of the above mentioned pathological conditions.

4.2.1.6 Amounts and Ratios

Provided herein are oral pharmaceutical preparations comprising varying amounts of a salt or polymorph of compound 101.

In certain embodiments, the pharmaceutical compositions comprise varying amounts of the besylate salt of compound 101.

In certain embodiments, the besylate salt of compound 101 is present in an amount of about 0.1 to about 20.0 mg, about 0.5 to about 10.0 mg, about 0.5 to about 5.0 mg, about 0.5 to about 4.0 mg, about 0.5 to about 3.0 mg, about 0.5 to about 2.0 mg, about 0.5 to about 1.0 mg, about 1.0 to about 2.0 mg, about 2.0 to about 3.0 mg, about 3.0 to about 4.0 mg, about 4.0 to about 5.0 mg, and about 5.0 to about 10.0 mg per capsule.

In certain embodiments, the besylate salt of compound 101 is present in an amount of about 0.1 mg, about 0.5 mg, about 1.0 mg, about 2.0 mg, about 3.0 mg, about 4.0 mg, about 5.0 mg, and about 10.0 mg per capsule.

In certain embodiments, the oil-based medium is present in a volume of about 0.25 ml to about 0.75 ml, about 0.35 to about 0.45 ml, 0.25 to about 0.30 ml, about 0.30 to about 0.35 ml, about 0.35 to about 0.40 ml, about 0.40 to about 0.50 ml, about 0.50 to about 0.55 ml, about 0.55 to about 0.60 ml, about 0.60 to about 0.65 ml, about 0.65 to about 0.70 ml, and about 0.70 to about 0.75 ml per capsule

In certain embodiments, the oil-based medium is present in a volume of about 0.25 ml, about 0.30 ml, about 0.35 ml, about 0.40 ml, about 0.45 ml, about 0.50 ml, about 0.55 ml, about 0.60 ml, about 0.65 ml, and about 0.75 ml per capsule.

Provided herein are also pharmaceutical compositions comprising varying amounts of a salt or polymorph of compound 101 and varying amounts of an oil-based medium. In certain embodiments, the oil-based medium comprises members that are present in varying amounts. In particular embodiments, the besylate salt of compound 101 and the members of the oil-based medium are present in varying amounts.

In exemplary embodiments, the amount of the besylate salt of compound 101 is about 0.65 mg, the amount of the Miglyol® 812N is about 209.6 mg, and the amount of the Phospholipon® 90 G is about 139.7 mg per capsule.

In exemplary embodiments, the amount of the besylate salt of compound 101 is about 1.31 mg, the amount of the Miglyol® 812N is about 209.2 mg, and the amount of the Phospholipon® 90 G is about 139.5 mg per capsule.

In exemplary embodiments, the amount of the besylate salt of compound 101 is about 2.62 mg, the amount of the Miglyol® 812N is about 208.4 mg, and the amount of the Phospholipon® 90 G is about 139.0 mg per capsule.

In exemplary embodiments, the amount of the besylate salt of compound 101 is about 3.92 mg, the amount of the Miglyol® 812N is about 207.6 mg, and the amount of the Phospholipon® 90 G is about 138.4 mg per capsule.

In exemplary embodiments, the amount of the besylate salt of compound 101 is about 0.65 mg per capsule. In exemplary embodiments, the amount of the Miglyol® 812N is about 209.6 mg per capsule. In exemplary embodiments, the amount of the Phospholipon® 90 G is about 139.7 mg per capsule.

In exemplary embodiments, the amount of the besylate salt of compound 101 is about 1.31 mg per capsule. In exemplary embodiments, the amount of the Miglyol® 812N is about 209.2 mg per capsule. In exemplary embodiments, the amount of the Phospholipon® 90 G is about 139.5 mg per capsule.

In exemplary embodiments, the amount of the besylate salt of compound 101 is about 2.62 mg per capsule. In exemplary embodiments, the amount of the Miglyol® 812N is about 208.4 mg per capsule. In exemplary embodiments, the amount of the Phospholipon® 90 G is about 139.0 mg per capsule.

In exemplary embodiments, the amount of the besylate salt of compound 101 is about 3.92 mg per capsule. In exemplary embodiments, the amount of the Miglyol® 812N is about 207.6 mg per capsule. In exemplary embodiments, the amount of the Phospholipon® 90 G is about 138.4 mg per capsule.

Provided further herein are pharmaceutical compositions comprising varying ratios of a salt or polymorph of compound 101 to an oil-based medium. In certain embodiments, the members of the oil-based medium are present in varying ratios. In particular embodiments, the besylate salt of compound 101 and the members of the oil-based medium are present in varying ratios.

In exemplary embodiments, the besylate salt of compound 101 is present in a wt/wt % of about 0.19%, the Miglyol® 812N is present in a wt/wt % of about 59.9% and the Phospholipon® 90 G is present in a wt/wt % of about 39.9%, per capsule.

In exemplary embodiments, the besylate salt of compound 101 is present in a wt/wt % of about 0.37%, the Miglyol® 812N is present in a wt/wt % of about 59.8%, and the Phospholipon® 90 G is present in a wt/wt % of about 39.9% per capsule.

In exemplary embodiments, the besylate salt of compound 101 is present in a wt/wt % of about 0.75%, the Miglyol® 812N is present in a wt/wt % of about 59.6%, and the Phospholipon® 90 G is present in a wt/wt % of about 39.7% per capsule.

In exemplary embodiments, the besylate salt of compound 101 is present in a wt/wt % of about 1.12%, the Miglyol® 812N is present in a wt/wt % of about 59.3%, and the Phospholipon® 90 G is present in a wt/wt % of about 39.6% per capsule.

In exemplary embodiments, the besylate salt of compound 101 is present in a wt/wt % of about 0.19% per capsule. In exemplary embodiments, the Miglyol® 812N is present in a wt/wt % of about 59.9% per capsule. In exemplary embodiments, the Phospholipon® 90 G is present in a wt/wt % of about 39.9% per capsule.

In exemplary embodiments, the besylate salt of compound 101 is present in a wt/wt % of about 0.37% per capsule. In exemplary embodiments, the Miglyol® 812N is present in a wt/wt % of about 59.8% per capsule. In exemplary embodiments, the Phospholipon® 90 G is present in a wt/wt % of about 39.9% per capsule.

In exemplary embodiments, the besylate salt of compound 101 is present in a wt/wt % of about 0.75% per capsule. In exemplary embodiments, the Miglyol® 812N is present in a wt/wt % of about 59.6% per capsule. In exemplary embodiments, the Phospholipon® 90 G is present in a wt/wt % of about 39.7% per capsule.

In exemplary embodiments, the besylate salt of compound 101 is present in a wt/wt % of about 1.12% per capsule. In exemplary embodiments, the Miglyol® 812N is present in a wt/wt % of about 59.3% per capsule. In exemplary embodiments, the Phospholipon® 90 G is present in a wt/wt % of about 39.6% per capsule.

4.2.2 Methods of Treatment

In yet another aspect, provided herein are methods of treating PPARγ-mediated conditions or diseases by administering to a subject having such a disease or condition, a therapeutically effective amount of an oral pharmaceutical preparation comprising a salt or polymorph of compound 101, as provided herein. The subject can be an animal such as, for example, a mammal, including, but not limited to, a primate (e.g., a human), a cow, a sheep, a goat, a horse, a dog, a cat, a rabbit, a rat, a mouse, and the like.

Depending on the biological environment (e.g., cell type, pathological condition of the host, etc.), these oral pharmaceutical preparations can activate or block the actions of PPARγ. By activating, i.e., agonizing, the PPARγ receptor, the oral pharmaceutical preparations will find use as therapeutic agents capable of modulating conditions mediated by the PPARγ receptor. As noted above, examples of such conditions include type II diabetes. Thus, PPARγ receptor agonists can be used to treat conditions including type II diabetes. Additionally, the oral pharmaceutical preparations can be useful for the prevention and treatment of complications of diabetes (e.g., neuropathy, retinopathy, glomerulosclerosis, and cardiovascular disorders), and preventing or treating hyperlipidelinia. Still further, the oral pharmaceutical preparations can be useful for the modulation of inflammatory conditions which most recently have been found to be controlled by PPARγ. (See, Ricote et al., 1998, Nature 391:79-82, and Jiang et al., 1998, Nature 391:82-86.) Examples of inflammatory conditions include rheumatoid arthritis and atherosclerosis. Oral pharmaceutical preparations that act via antagonism of PPARγ can be useful for treating obesity, hypertension, hyperlipidemia, hypercholesterolemia, hyperlipoproteinemia, and metabolic disorders.

In therapeutic use for the treatment of obesity, diabetes, inflammatory conditions or other conditions or disorders mediated by PPARγ, the salts or polymorphs of compound 101 can be administered as the oral pharmaceutical preparations described herein at the initial dosage of about 0.001 mg to about 100 mg daily. A daily dose range of about 0.1 mg to about 10 mg is preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

In the treatment or prevention of conditions which require PPARγ receptor modulation an appropriate dosage level will generally be about 0.001 to 100 mg salt or polymorph of compound 101 per day which can be administered in single or multiple doses of the oral pharmaceutical preparation provided herein. Preferably, the dosage level will be about 0.01 to about 25 mg per day; more preferably about 0.05 to about 10 mg per day. A suitable dosage level may be about 0.01 to 25 mg per day, about 0.05 to 10 mg per day, or about 0.1 to 5 mg per day. Within this range the dosage may be 0.005 to 0.05, 0.05 to 0.5, or 0.5 to 5.0 mg per day. The oral pharmaceutical preparations provided herein are preferably provided in the form of ingestible capsules containing 0.1 to 20 milligrams of the salt or polymorph of compound 101, particularly 0.1, 0.2, 0.3, 0.4, 0.5, 0.6, 0.7, 0.8, 0.9, 1.0, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.2, 4.4, 4.5, 4.6, 4.8, 5.0, 5.5, 6.0, 6.5, 7.0, 7.5, 8.0, 8.5, 9.0, 9.5, 10.0, or 20.0 mg of the salt or polymorph of compound 101 for the symptomatic adjustment of the dosage to the patient to be treated. The oral pharmaceutical preparations provided herein may be administered on a regimen of 1 to 4 times per day, preferably once or twice per day.

It will be understood, however, that the specific dose level and frequency of dosage for any particular patient may be varied and will depend upon a variety of factors including the activity of the specific polymorph employed, the metabolic stability and length of action of that polymorph, the age, body weight, general health, sex, diet, mode and time of administration, rate of excretion, drug combination, the severity of the particular condition, and the host undergoing therapy.

The oral pharmaceutical preparations described herein can be combined with other compounds having related utilities to treat or prevent metabolic disorders and inflammatory conditions, complications thereof and pathologies associated therewith (e.g., cardiovascular disease and hypertension). In many instances, administration of the subject oral pharmaceutical preparations in conjunction with these alternative agents enhances the efficacy of such agents. Accordingly, in some instances, the present oral pharmaceutical preparations, when combined or administered in combination with, e.g., anti-diabetic agents, can be used in dosages which are less than the expected amounts when used alone, or less than the calculated amounts for combination therapy.

Thus, provided herein is a single capsule unit dosage form comprising: (a) compound 101, or a salt or polymorph thereof; (b) an oil-based medium; and (c) an alternative agent. In one aspect, the PPARγ modulator is added in a solid form during the manufacturing process and is dissolved in an oil-based medium.

For example, suitable agents for combination therapy include those that are currently commercially available and those that are in development or will be developed. Exemplary agents useful in the treatment of metabolic disorders include, but are not limited to: (a) anti-diabetic agents such as insulin, sulfonylureas (e.g., meglinatide, tolbutamide, chlorpropamide, acetohexamide, tolazamide, glyburide, glipizide, and glimepiride), biguanides, e.g., metformin (Glucophage®), a-glucosidase inhibitors (acarbose), thiazolidinone compounds, e.g., rosiglitazone (Avandia®, troglitazone (Rezulin®), and pioglitazone (Actos®); (b)β₃ adrenergic receptor agonists, leptin or derivatives thereof, and neuropeptide Y antagonists; (c) bile acid sequestrants (e.g., cholestyramine and colestipol), HMG-CoA reductase inhibitors, e.g., statins (e.g., lovastatin, atorvastatin, fluvastatin, pravastatin and simvastatin), nicotinic acid (niacin), fibric acid derivatives (e.g., gemfibrozil and clofibrate), and nitroglycerin. Exemplary agents useful in the treatment of inflammatory conditions include, but are not limited to: (a) non-steroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (e.g., flufenamic acid, meclofenamic acid, mefenamic acid, niflumic acid, and tolfenamic acid), biphenylcarboxylic acid derivatives (e.g., diflunisal and flufenisal), oxicams (e.g., isoxicam, piroxicam, sudoxicam, and tenoxican), salicylates (e.g., acetyl salicylic acid and sulfasalazine) and the pyrazolones (e.g., apazone, bezpiperylon, feprazone, mofebutazone, oxyphenbutazone, and phenylbutazone); (b) cyclooxygenase-2 (COX-2) inhibitors such as celecoxib (Celebrex®) and rofecoxib (Vioxx®); (c) inhibitors of phosphodiesterase type IV (PDE-IV); and (d) inhibitors of the dipeptidyl peptidase-IV (DPP-IV) enzyme such as vildagliptin, sitagliptin, saxagliptin, PSN9301, SYR 322, SYR 472, dipeptide derivatives or dipeptide mimetics (e.g., alanine-pyrrolidide, isoleucine-thiazolidide, and the pseudosubstrate N-valyl prolyl, O-benzoyl hydroxylamine), β-aminoacid derivatives (e.g., 3(R)-Amino-1-[3-(trifluoromethyl)-5,6,7,8-tetrahydro[1,2,4]triazolo[4,3-a-]pyrazin-7-yl]-4-(2,4,5-trifluorophenyl)butan-1-one (MK-0431)), cyanopyrrolidides (e.g., (1-[[3-hydroxy-1-adamantyl)amino]acetyl]-2-cyano-(S)-pyrrolidine (LAF237 or vildagliptin), 1-[2-[5-cyanopyridin-2-yl)amino]ethylamino]acetyl-2-cyano-(S)-pyrrolidine (e.g., NVP-DPP728), (1 S,3 S,5 S)-2-[2(S)-Amino-2-(3-hydroxyadamantan-1-yl)acetyl]-2-azabicyclo[-3.1.0]hexane-3-carbonitrile (e.g., saxagliptin or BMS-47718), NVP-DPP728, 3-(L-Isoleucyl)thiazolidine (e.g., isoleucine-thiazolidide or PSN-9301), valine-pyrrolidides, [1-[2(S)-Amino-3-methylbutyryl]pyrrolidin-2(R)-yl]boronic acid (e.g., PT-100), and β-phenethylamines.

The weight ratio of the polymorph of compound 101 to the second active ingredient may be varied and will depend upon the effective dose of each ingredient. Generally, an effective dose of each will be used. Thus, for example, when a polymorph of compound 101 is combined with an NSAID the weight ratio of the polymorph to the NSAID will generally range from about 1000:1 to about 1:1000, preferably about 200:1 to about 1:200. Combinations of a salt or polymorph of compound 101 and other active ingredients will generally also be within the aforementioned range, but in each case, an effective dose of each active ingredient should be used.

In certain embodiments, the oral pharmaceutical preparations disclosed herein may be used to treat or prevent a variety of other indications. Such indications include, but are not limited to, metabolic conditions such as diabetes (including type I and type II diabetes), hypertension, angina pectoris, dyslipidemia (including hypertriglyceridemia, hyperlipoproteinemia, and hypercholesterolemia), gout, nephropathy and other renal diseases secondary to diabetes, diabetic neuropathy, other insulin-resistance-related diseases, polycystic ovarian syndrome, glucocorticoid-induced insulin resistance, obesity, bone disorders, female-specific conditions (including excessive climacteric uterine bleeding), and acne; neurological disorders such as Alzheimer's disease, neuroinflammation, ischemic stroke, closed-head injury, and multiple sclerosis; proliferative disorders such as atherosclerosis, restenosis, colon cancer, prostate cancer, breast cancer, liposarcoma, epithelial cell cancers, uroepithelial cancer, and other cancers; and inflammatory or immune disorders such as rheumatoid arthritis, inflammatory bowel disease, colitis, Crohn's disease, macular degeneration, other inflammatory disorders, and other immune disorders. Rationales suggesting the utility of the oral pharmaceutical preparations of the present provided herein for treating or preventing such indications are discussed in detail in U.S. Pat. No. 7,223,761.

In particularly preferred embodiments, the oral pharmaceutical preparations provided herein are directed to the treatment or prevention of type II diabetes using a salt or polymorph of compound 101, either alone or in combination with a second therapeutic agent selected from anti-diabetic agents such as insulin, sulfonylureas (e.g., meglinatide, tolbutamide, chlorpropamide, acetohexamide, tolazamide, glyburide, glipizide, and glimepiride), biguanides, e.g., metformin (Glucophage®), a-glucosidase inhibitors (acarbose), thiazolidinone compounds, e.g., rosiglitazone (Avandia®, troglitazone (Rezulin®), and pioglitazone (Actos®). When used in combination, the practitioner can administer a combination of the therapeutic agents, or administration can be sequential.

4.2.3 Methods of Making

In yet another aspect, provided herein are methods of making the oral pharmaceutical preparations provided herein. To make the oral pharmaceutical preparations, a capsule body, for example, a hard capsule body, is filled with a pharmaceutical composition, a capsule cap, for example, a hard capsule cap, is secured onto the capsule body containing the composition to form a liquid-filled capsule, and then the capsule is sealed, e.g., by spraying a small amount of water/ethanol mixture at the cap and body interface and/or followed by gelatin banding to fuse the two capsule parts together.

In certain embodiments, provided herein are methods of making an oral pharmaceutical preparations comprising the steps of: (i) filling a hard capsule body with a composition comprising compound 101, or salt thereof, in an oil-based medium; (ii) securing a cap onto the capsule body containing the composition to form a filled capsule; and (iii) sealing the capsule with a gelatin band.

In particular embodiments, the oil-based medium comprises a caprylic/capric acid and a phosphatidylcholine.

The oil-based medium comprising a caprylic/capric acid and a phosphatidylcholine may be made, for example, by mixing caprylic/capric acid and phosphatidylcholine at 65-70° C. with stirring until the phosphatidylcholine is completely dissolved. Typically, about 2 hours of stirring at 65-70° C. result in dissolution of the phosphatidylcholine.

In another embodiment, a pre-made mixture of oil-based medium can be used. For example, Phosal® 53 MCT or LIPOID S 75-MCT (30% LIPOID S 75 and 70% MCT) mixtures can be used for the purposes of the present invention.

In particular embodiments, the composition comprises a salt or polymorph of compound 101 in an oil-based medium comprising caprylic/capric acid and phosphatidylcholine.

The composition comprising a salt or polymorph of compound 101 in an oil-based medium comprising caprylic/capric acid and phosphatidylcholine may be made, for example, by adding to a completely dissolved caprylic/capric acid and phosphatidylcholine mixture to a salt or polymorph of compound 101 and sonicating the resulting mixture until the salt or polymorph of compound 101 is completely dissolved.

In particular embodiments, the capsule comprises gelatin, plasticized gelatin, hydroxypropylmethylcellulose, starch or agar. In certain embodiments, the capsule comprises gelatin.

In certain embodiments, the capsule comprises soft gelatin.

In a preferred embodiment, the capsule comprises hard gelatin.

As described above, hard capsules can be produced from the capsule material using conventional techniques known to those of ordinary skill in the art. See, for example, U.S. Pat. No. 4,917,885, U.S. Pat. No. 5,431,917, U.S. Pat. No. 6,413,463, and U.S. Pat. No. 6,649,180.

As described above, plasticizers may be added to the capsule material to increase the flexibility and strength.

Once the capsule body and cap are formed, the composition comprising the salt or polymorph compound 101 in the oil-based medium is metered out and placed into the capsule body. The capsule cap is then secured onto the capsule body to form a filled capsule having an interior volume.

As described above, the filling of the capsule body can be performed using any capsule-filling technique known to those skilled in the art. One machine for industrial filling of capsules is commercially available and is marketed under the name LIQFIL (Qualicaps F-40 LIQFIL Super 40, fabricated by Qualicaps, Inc.).

Once the capsule body is filled with the pharmaceutical composition, and the capsule cap is secured onto the capsule body to faun a liquid-filled capsule, the joined capsule halves can be banded at their seam of overlap with a gelatin band. As described above, the gelatin band may be placed around the capsule in a solution form and then cooled, thereby sealing the capsule. One machine for industrially sealing capsules with a gelatin band is commercially available and is marketed under the name HICAPSEAL (Qualicaps S-40 HICAPSEAL, fabricated by Qualicaps, Inc.).

As described above, the capsules provided herein may also be coated with an enteric coating, alone or in addition to another coating.

The enteric coating may be applied to the capsule by press coating, pan coating, molding, spraying, dipping and/or air-suspension or air tumbling procedures, or any other procedure known to those of skill in the art. The enteric coating material and solvent should be compatible with the capsule material and any other coating. Solvents suitable for applying the enteric coating include an alcohol, ketone, ester, ether, aliphatic hydrocarbon, halogenated solvents, cycloaliphatic solvents, aromatic, heterocyclic, aqueous solvents, and mixtures thereof.

As described above, the capsule may additionally be coated with a controlled release coating, compatible with the other components of the enteric coating. The coating may be applied in the form of an organic or aqueous solution or dispersion.

### 5. EXAMPLES

Reagents and solvents used below can be obtained from commercial sources such as Aldrich Chemical Co. (Milwaukee, Wis., USA). ¹H-NMR spectra were recorded on a Varian Gemini 400 MHz NMR spectrometer. Significant peaks are tabulated in the order: number of protons, multiplicity (s, singlet; d, doublet; t, triplet; q, quartet; m, multiplet; br s, broad singlet) and coupling constant(s) in Hertz (Hz). Electrospray ionization (ESI) mass spectrometry analysis was conducted on a Hewlett-Packard 1100 MSD electrospray mass spectrometer using the HP 1100 HPLC for sample delivery.

Mass spectrometry results are reported as the ratio of mass over charge. The compound was dissolved in methanol at 0.1 mg/mL and 1 microliter was infused with the delivery solvent into the mass spectrometer, which scanned from 100 to 1500 daltons. The compound could be analyzed in the positive ESI mode, using 1:1 acetonitrile/water with 1% acetic acid as the delivery solvent. The compound could also be analyzed in the negative ESI mode, using 2 mM NH₄OAc in acetonitrile/water as delivery solvent.

### 5.1 Example 1

### Synthesis of Compound 101

This example provides an exemplary synthesis of compound 101. Alternate methods of synthesizing compound 101, including methods of synthesizing acid addition salts of compound 101 are described below; still other alternate synthetic methods will be apparent to those of skill in the art.

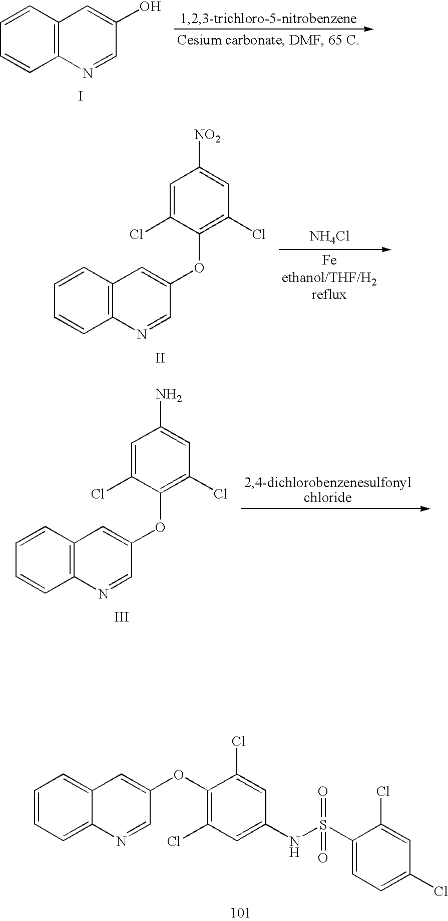

### 3-(2,6-Dichloro-4-nitro-phenoxy)-3,4-dihydro-quinoline (II)

3-Hydroxyquinoline (I) (prepared according to the procedure of Naumann et. al., Synthesis 4:279-281 (1990)) (3 g) and 1,2,3-trichloro-5-nitrobenzene (4.7 g) were dissolved in DMF (80 mL) and heated with cesium carbonate (7.4 g) for 2 h at 60° C. The reaction was poured into ice/water (500 mL). The resulting off-white precipitate was collected by filtration and rinsed with hexane to afford compound II as a solid (6.9 g) suitable for use in the next reaction.

¹H NMR in CDCl₃ d 8.863 (d, J=2.2 Hz, 1H), 8.360 (s, 2H), 8.106 (d, J=8.6 Hz, 1H), 7.646 (m, 2H), 7.529 (d, J=8.6 Hz, 1H), 7.160 (d, J=2.2 Hz, 1H).

### 3,5-Dichloro-4-(3,4-dihydro-quinolin-3-yloxy)-phenylamine (III)

To a solution of compound II (6.9 g) in ethanol/THF/water (ratio 40:20:10) was added ammonium chloride (3.3 g) and powdered iron (3.4 g). This mixture was heated to reflux for 5 h. The hot mixture was then filtered through Celite and concentrated. The residue was dissolved in ethyl acetate and washed with saturated NaHCO₃ solution followed by water and then brine. The solution was dried over magnesium sulfate and concentrated to afford compound III as an off-white solid (5.6 g).

¹H NMR in (DMSO) d 8.846 (d, J=2.9 Hz, 1H), 8.010 (m, 1H), 7.915 (m, 1H), 7.645 (m, 1H), 7.560 (m, 1H), 7.401 (d, J=2.9 Hz, 1H), 6.778 (s, 2H), 5.762 (s, 2H).

### 2,4-Dichloro-N-[3,5-dichloro-4-(quinolin-3-yloxy)-phenyl]-benzenesulfonamide (101)

Treatment of the aniline III with 2,4-dichlorobenzenesulfonyl chloride according to conventional methods gave compound 101.

¹H NMR (d₆-acetone) d 9.9 (1H, br s), 8.794 (1H, d, J=2.9 Hz), 8.23 (1H, d, J=8.4 Hz), 8.035 (1H, br d, J=8.4 Hz), 7.793 (1H, d, J=1.5 Hz), 7.78 (1H, m), 7.62-7.70 (2H, m), 7.57 (1H, td, J=6.8, 1.2 Hz), 7.476 (2H, s), 7.364 (1H, d, J=2.6 Hz). MS (M-H) 511.0.

### 5.2 Example 2

### PPARγ Ligand Binding

Using methods similar to Lehmann et al., J. Biol. Chem. 270:12953-12956 (1995), compound 101, prepared according to Example 1, exhibited an IC₅₀ of less than 1 μM in a PPARγ ligand binding assay utilizing [³H]-BRL 49653 as the radioligand.

### 5.3 Example 3

### Large Scale Synthesis of the Besylate Salt of Compound 101

This example provides an exemplary synthesis of the besylate salt of compound 101 from precursors to compound 101. Alternate methods of synthesizing the besylate salt of compound 101 from such precursors will be apparent to those of skill in the art.

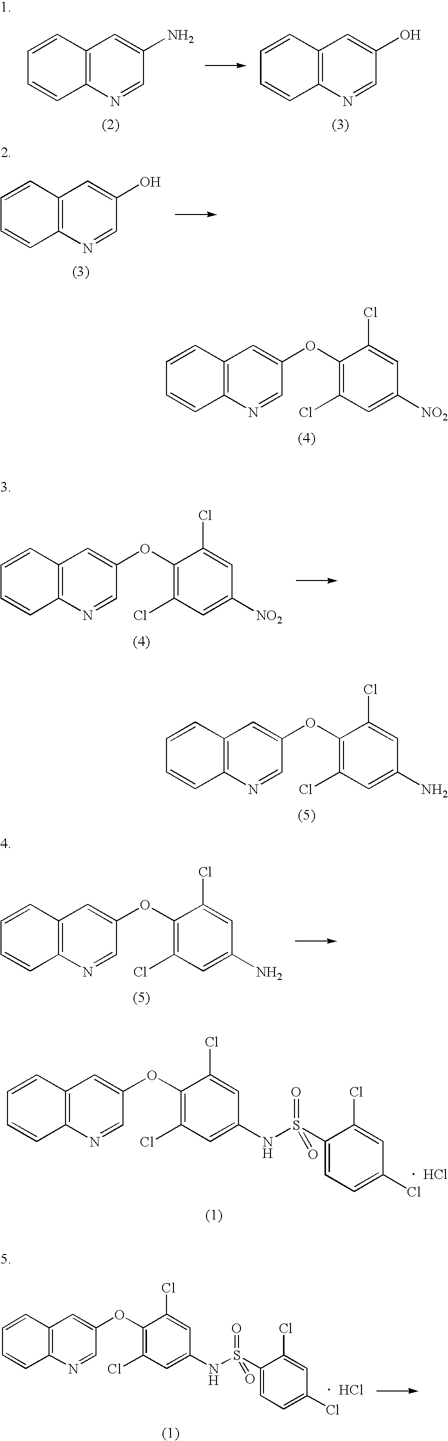

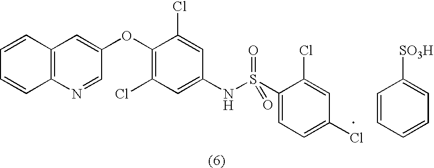

### Synthesis of Salts of Compound 101 According to Examples 3 and 4

### 3-hydroxyquinoline (3)

3-aminoquinoline (2), via the diazonium salt, was converted to 3-hydroxyquinoline (3) in 96% yield.

### 3-(2,6-Dichloro-4-nitro-phenoxy)-quinoline (4)

3-Hydroxyquinoline (3) and 1,2,3-trichloro-5-nitrobenzene were dissolved in DMF and heated with calcium carbonate to give, after titration with isopropanol, 3-(2,6-dichloro-4-nitro-phenoxy)-quinoline (4) in 93% yield.

### 3,5-dichloro-4-(quinolin-3-yloxy)-phenylamine (5)

The nitro functionality of 3-(2,6-dichloro-4-nitro-phenoxy)-quinoline (4) was catalytically reduced under hydrogen with 5% weight/weight (catalyst/compound 4) of a 1% platinum/2% vanadium on carbon catalyst suspension in ethyl acetate at 0° C. The material was heated to 20° C., filtered through Celite. The Celite was washed with THF, and the filtrates were combined and evaporated to give 3,5-dichloro-4-(quinolin-3-yloxy)-phenylamine (5) in 98% yield.

### 2,4-dichloro-N-[3,5-dichloro-4-quinolin-3-yloxy)phenyl]-benzenesulfonamide HCl (1)

3,5-dichloro-4-(quinolin-3-yloxy)-phenylamine (5) was then reacted with 2,4-dichlorobenzenesulfonylchloride, followed by treatment with hydrochloric acid, to give 2,4-dichloro-N-[3,5-dichloro-4-quinolin-3-yloxy)phenyl]-benzenesulfonamide HCl (1; the hydrochloride salt of compound 101) in 99% yield.

### 5.4 Example 4

### Preparation of the Besylate Salt of Compound 101

The besylate salt of compound 101 was synthesized from 2,4-dichloro-N-[3,5-dichloro-4-quinolin-3-yloxy)phenyl]-benzenesulfonamide HCl prepared according to Example 3. The hydrochloride salt 2,4-dichloro-N-[3,5-dichloro-4-quinolin-3-yloxy)phenyl]-benzenesulfonamide HCl was converted to the besylate salt, via the free base, using a sodium bicarbonate/ethyl acetate biphasic reaction solution. Separation of the organic layer followed by solvent exchange with ethanol precipitated the besylate salt (6) of compound 101 in 84% yield. Starting from 4-aminoquinoline (2), the overall yield of the besylate salt (6) of compound 101 was 73%.

The preparation described in Examples 3 and 4 was performed two times; one batch yielded a mixture of Forms I and II of the besylate salt of compound 101. The other batch yielded only the Form II polymorph of the besylate salt of compound 101.

### 5.5 Example 5

### Recrystallization of Form II of the Besylate Salt of Compound 101

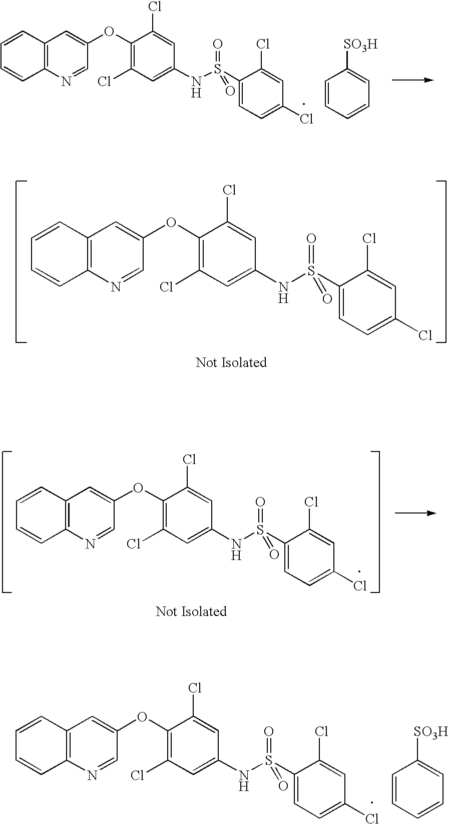

Compound 101 was recrystallized as Form II with benzenesulfonic acid (PhSO₃H-xH₂O; Aldrich).

A mixture of Forms I and II of the besylate salt (6) of compound 101 (6.938 kg), prepared according to Examples 3 and 4, was stirred in ethyl acetate (115 L) with gentle heating (about 28° C.). A saturated solution of sodium bicarbonate (13 L) was added in portions (endothermic, gas evolution). The biphasic mixture was stirred for approximately 1 hour. The phases were separated and the organic layer washed with a saturated sodium chloride solution (13 L). The organic layer was separated and concentrated by distillation (91 L distillate removed). Ethyl acetate (91 L) was added, the solution decolorized with activated charcoal, then filtered through Celite. The filter cake was washed with ethyl acetate (2×15 L) and the filtrates combined with the ethyl acetate filtrate from the activated charcoal decolorizing step. The solution was concentrated by distilling off approximately 135 L. Ethanol (16 L) was added and the solution heated to 77° C. Benzenesulfonic acid (4.126 kg) dissolved in ethanol (5 L) was added. An additional 2 L of ethanol was used to rinse the vessel containing the benzenesulfonic acid solution. After cooling to approximately 69° C., 36 g of besylate salt (6) of compound 101 was added. The suspension was stirred at approximately 67 to 69° C. for 38 minutes, then cooled to 20° C. and stirred for approximately 4 hours. 6.377 kg (92%) solid was obtained after filtering and drying under vacuum.

### 5.6 Example 6

### Solubility of the Besylate Salt of Compound 101 in Selected Oil-Based Media

This example illustrates the solubility of the besylate salt of compound 101 in selected oil-based media.

The solubility of the besylate salt of compound 101 in selected oil-based media is provided in Table 1 below as concentration in mg/mL.

TABLE 1
Solubility of the besylate salt of compound 101 in selected
oil-based media

| | Concentration |
|---|---|
| Oil-based media | (mg/mL) |
| Labrasol ® | 29.18 |
| Labrafil ® M 1944 CS | 2.34 |
| Peceol | 3.94 |
| Labrafac ® CC | 0.29 |
| Super-refined soybean oil | 0.04 |
| Crodamol ® | 0.45 |
| Corn Oil | 0.16 |
| Capmul ® PG8 | 2.01 |
| Captex ® 300EP | 0.28 |
| Captex ® 200 | 0.45 |
| Captex ® 355 | 0.52 |
| Capmul ® MCM | 16.63 |
| Miglyol ® 812N | 7.93 |
| Phosal ® 53 MCT | 43.40 |
| Inwitor 742 | 7.93 |
| Miglyol ® 810N | 0.38 |
| Miglyol ® 132 | 0.80 |
| Maisine 35-I | 1.16 |

As can be seen in Table 1, solubility of the besylate salt of compound 101 is surprisingly high in phosphatidylcholine (e.g., Phosal® 53 MCT) and/or caprylic/capric acid (e.g., Capmul® MCM and Labrasol®) oil-based media.

### 5.7 Example 7

### Chemical Stability of the Besylate Salt of Compound 101 in Selected Oil-Based Media

This example illustrates the chemical stability of the besylate salt of compound 101 in the selected oil-based media of Example 6. Studies were performed over a 2 week period at −20° C., 2-8° C., 40° C./75% RH and 60° C. At the elevated temperatures, chemical stability was monitored in the presence and absence of argon. Additionally, the pH stability of the drug was assessed over a seven week period at 40° C./75% RH, pH 2, 3, 5, 7, 9, and 10.

In the presence of Labrasol®, Capmul® MCM, and Phospholipon® 90G, little or no degradation of the drug was observed at 40° C. over a 2-week period. In oil-based media containing neat Capmul® MCM or phosphatidylcholine (Phospholipon® 90G, Phosal® 53 MCT), drug recovery was >99%. Even at 60° C., no drug degradation was observed in Phosal® 53 MCT, and a small amount of degradation (7%) was observed in Capmul® MCM. The degradation observed in Capmul® MCM was inhibited by the addition of argon.

### 5.8 Example 8

### Physical Stability of the Besylate Salt of Compound 101 in Selected Oil-Based Media

This example illustrates the physical stability of the besylate salt of compound 101 in the selected oil-based media of Example 6.

Physical stability of the drug was monitored visually. Warming and then cooling the compositions in the presence of more than 50% Phospholipon® 90G resulted in the formation of a fine suspension. The addition of 47% Capmul® MCM to oil-based media that contained Phospholipon® 90G inhibited solid formation. High concentrations of Capmul® (>80%) caused solidification and cloudiness.

### 5.9 Example 9

### Formulation Examples

This example illustrates formulations for four strengths of the besylate salt of compound 101, which have surprising solubility, chemical stability, and physical stability, based on the studies of Examples 7 and 8. These four formulations, presented in Table 2, below, were made and tested (see Example 10, below).

TABLE 2
Formulations for four strengths of the besylate salt of compound 101

| | | Capsule Strength | | | | | | | |
|---|---|---|---|---|---|---|---|---|---|
| | | 0.5 mg | | 1 mg | | 2 mg | | 3 mg | |
| Component | Function | % | mg | % | mg | % | mg | % | mg |
| Besylate salt of | Active | 0.19 | 0.654* | 0.37 | 1.31* | 0.75 | 2.62* | 1.12 | 3.92* |
| compound 101 | component | | | | | | | | |
| Phosphatidyl-choline | Oil-based | 39.9 | 139.7 | 39.9 | 139.5 | 39.7 | 139.0 | 39.6 | 138.4 |
| (Phospholipon ® 90 G) | medium | | | | | | | | |
| Caprylic/capric | Oil-based | 59.9 | 209.6 | 59.8 | 209.2 | 59.6 | 208.4 | 59.3 | 207.6 |
| triglyceride | medium | | | | | | | | |
| (Miglyol ® 812N) | | | | | | | | | |
| Capsule, white opaque, | Hard | NA | 1 ea | NA | 1 ea | NA | 1 ea | NA | 1 ea |
| Size 1 NF4186 | capsule | | | | | | | | |
| Clear gelatin banding | Sealing | NA | q.s. | NA | q.s. | NA | q.s. | NA | q.s. |
| solution** | | | | | | | | | |

NA—not applicable
*Quantities are for the besylate salt of compound 101 are equivalent to 0.5, 1.0, 2.0, and 3.0 mg/capsule compound 101 free base.
**Contains 21.69% gelatin, 0.92% polysorbate 80 NF/PhEur/JP, 77.39% purified water.

### 5.10 Example 10

### Stability Protocol for Testing Formulations

This example illustrates the stability protocol for testing the stability specifications for the four formulations of Example 9. The formulations were tested for stability at several storage conditions, and at time points extending to 24 months. The longest periods for which the formulations were tested for stability are as follows:

5° C.—six months;

25° C./60% RH—22 months;

30° C./65% RH—15 months; and

40° C./75% RH—15 months.

### 5.11 Example 11

### Specifications for Formulations

This example illustrates the release and stability specifications for the formulations of Example 9, as provided in Table 3 below.

TABLE 3
Specifications for tested formulations

| Parameter | Release Specification | Stability Specification |
|---|---|---|
| Appearance | White opaque, size 1, liquid filled | Same as release specification |
| | gelatin capsules containing | |
| | slightly yellowish liquid or | |
| | viscous liquid or gel | |
| Identification | | |
| HPLC | Sample retention time | Not tested |
| | corresponds to reference standard | |
| | within 0.5 minutes | |
| UV spectrum | Sample UV spectrum corresponds | |
| | to reference standard from 200 to | |
| | 300 nm | |
| Potency by HPLC | 95.0-105.0 | 90.0-110.0 |
| (% label claim) | | |
| Purity by HPLC | | |
| (area %) | | |
| Purity | =98.0% | Record |
| Related | =2.0 total, =0.5 individual | Record |
| substances | | |
| Dissolution | Q = 70 at 60 minutes | Same as release specification |
| Content Uniformity | Meets USP | Not tested |
| Microbial Limits Test | Total aerobic microbial count = | Same as release specification |
| | NMT 1000 CFU/g | |
| | Total combined yeasts and mold | |
| | count = 100 CFU/g | |
| | Absence of: Escherichia coli, | |
| | Salmonella, Pseudomonas | |
| | aeruginosa, Staphylococcus | |
| | aureus | |

The formulations of Example 9 meet the stability specifications when stored for up to 22 months at 25° C./60% RH, 15 months at 30° C./65% RH, and 15 months at 40° C./75% RH.

All publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those of ordinary skill in the art in light of the teachings of the specification that certain changes and modifications may be made thereto without departing from the spirit or scope of the appended claims.

What is claimed is:

1. An oral pharmaceutical preparation comprising:
(a) a compound of formula (I): 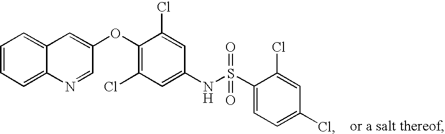
in an oil-based medium; and
(b) a hard capsule encapsulating the composition.

2. The oral pharmaceutical preparation of claim 1 wherein the composition comprises a salt of the compound of formula (I) selected from the group consisting of a benzenesulfonate, hydrochloride, hydrobromide, and p-toluenesulfonate salt.

3. The oral pharmaceutical preparation of claim 2, wherein the salt is benzenesulfonate.

4. The oral pharmaceutical preparation of claim 1, wherein the oil-based medium comprises one or more members selected from the group consisting of corn oil, super-refined soybean oil, Capmul® MCM, Capmul® PG8, Captex® 200, Captex® 300 EP, Captex® 355, Crodamol®, Inwitor 742, Labrafac® CC, Labrafil® M 1944 CS, Labrasol®, Peceol, Phosal® 53 MCT, Phospholipon® 90G, Maisine 35-I, Miglyol® 132, Miglyol® 810N, and Miglyol® 812N.

5. The oral pharmaceutical preparation of claim 3, wherein the benzenesulfonate salt of the compound of formula (I) is a Form I or Form II polymorph.

6. The oral pharmaceutical preparation of claim 5, wherein the benzenesulfonate salt of the compound of formula (I) is a Form II polymorph obtained by crystallizing the benzenesulfonate salt of the compound of formula (I) from ethanol.

7. The oral pharmaceutical preparation of claim 3, wherein the benzenesulfonate salt of the compound of formula (I) is present in an amount of about 0.1-20.0 mg per capsule.

8. The oral pharmaceutical preparation of claim 3, wherein the benzenesulfonate salt of the compound of formula (I) is present in an amount of about 0.5-3.0 mg per capsule.

9. The oral pharmaceutical preparation of claim 4, wherein the two members are Phospholipon® 90 G and Miglyol® 812N.

10. The oral pharmaceutical preparation of claim 9, wherein the Miglyol® 812N and Phospholipon® 90 G are present in a wt/wt ratio of about 60:40 of Miglyol® 812N to Phospholipon® 90 G.

11. The oral pharmaceutical preparation of claim 1 wherein the composition is a microemulsion.

12. The oral pharmaceutical preparation of claim 1, wherein the capsule comprises gelatin, plasticized gelatin, hydroxypropylmethylcellulose, starch or agar.

13. The oral pharmaceutical preparation of claim 12, wherein the capsule comprises gelatin.

14. The oral pharmaceutical preparation of claim 1, wherein the capsule is sealed using gelatin banding.

15. An oral pharmaceutical preparation comprising:
(a) a benzenesulfonate salt of the compound of formula (I): 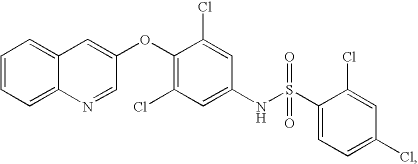
in an oil-based medium; wherein the oil-based medium comprises Phospholipon® 90 G and Miglyol® 812N; and
(b) a capsule encapsulating the composition.

16. The oral pharmaceutical preparation of claim 15, wherein the capsule comprises soft gelatin.

17. The oral pharmaceutical preparation of claim 15, wherein the capsule comprises hard gelatin.

18. The oral pharmaceutical preparation of claim 15, wherein the Miglyol® 812N and Phospholipon® 90 G are present in a wt/wt ratio of about 60:40 of Miglyol® 812N to Phospholipon® 90 G.

19. The oral pharmaceutical preparation of claim 15, wherein the amount of the benzenesulfonate salt of the compound of formula (I) is about 0.65 mg, the amount of the Miglyol® 812N is about 209.6 mg, and the amount of the Phospholipon® 90 G is about 139.7 mg per capsule.

20. The oral pharmaceutical preparation of claim 15, wherein the amount of the besylate salt of compound of formula (I) is about 1.31 mg, the amount of the Miglyol® 812N is about 209.2 mg, and the amount of the Phospholipon® 90 G is about 139.5 mg per capsule.

21. The oral pharmaceutical preparation of claim 15, wherein the amount of the benzenesulfonate salt of the compound of formula (I) is about 2.62 mg, the amount of the Miglyol® 812N is about 208.4 mg, and the amount of the Phospholipon® 90 G is about 139.0 mg per capsule.

22. The oral pharmaceutical preparation of claim 15, wherein the amount of the benzenesulfonate salt of the compound of formula (I) is about 3.92 mg, the amount of the Miglyol® 812N is about 207.6 mg, and the amount of the Phospholipon® 90 G is about 138.4 mg per capsule.

23. The oral pharmaceutical preparation of claim 15, wherein (i) the benzenesulfonate salt of the compound of formula (I) is present in an amount of about 1.0-20.0 mg per capsule; (ii) the Miglyol® 812N and Phospholipon® 90 G are present in a wt/wt ratio of about 60:40 of Miglyol® 812N to Phospholipon® 90 G; (iii) the oil-based medium is present in a volume of about 0.5 ml; (iv) the capsule comprises gelatin; (v) the capsule size is Size No. 1; and (vi) the capsule is sealed with gelatin banding.

24. A method of treating a PPARγ-mediated condition or disorder in a human, the method comprising administering to the subject a therapeutically effective amount of the oral pharmaceutical preparation of claim 1.
